(19) United States
(12) Patent Application Publication
(10) Pub. No.: US 2004/0191332 A1
(43) Pub. Date: Sep. 30, 2004

(54) Preserved ophthalmic compositions

(75) Inventors: James N. Chang, Newport Beach, CA (US); Richard Graham, Irvine, CA (US); Orest Olejnik, Trabuco Canyon, CA (US); Stanley W. Huth, Newport Beach, CA (US); Michelle Luu, Garden Grove, CA (US)

Correspondence Address: Frank J. Uxa, Stout, Uxa, Buyan & Mullins, LLP, Suite 300, 4 Venture, Irvine, CA, 92618, US

(73) Assignee: Allergan, Inc., Irvine, CA

(21) Appl. No.: 10/400,893

(22) Filed: Mar. 27, 2003

(51) Int. Cl. A61K 31/498; A61K 33/14

(52) U.S. Cl. 424/661; 514/249

(57) ABSTRACT

Ophthalmic compositions include a carrier component, an oxy-chloro component present at an amount effective in preserving the composition, and at least one additional component, e.g. a borate component and/or a glycerin component present in an amount effective to enhance a preservative efficacy of the composition. The compositions preferably also include one or more other components, such as therapeutic components, e.g., quinoxaline components, and polyanionic components effective to provide the compositions with one or more functionalities.

### BACKGROUND OF INVENTION

The present invention relates to preserved ophthalmic compositions and methods of preserving ophthalmic compositions. More particularly, the invention relates to ophthalmic compositions, including those useful for drug delivery to the eye, those to treat dry eye and otherwise care for the eye, contact lens care compositions and the like, which are benefited from being preserved.

Ophthalmic compositions often utilize at least one preservative, depending on the type of composition. Certain therapeutics included in such compositions are often sensitive to and may become inactivated by certain preservatives. This adverse effect can be minimized or eliminated in some cases if the preservative is present at a reduced concentration. In addition, such a reduced concentration of preservative may be advantageous in preventing eye irritation or other adverse effects that may be caused by certain preservatives. However, in some cases a reduced preservative concentration may produce a composition which does not pass certain standards such as the USP, EP-A and/or EP-B preservative efficacy tests or standards. Furthermore, preservatives may become inactivated over time, thus requiring a higher initial concentration of preservative. This inactivation may be facilitated by, for example, exposure to one or more ingredients included in a composition.

Various ophthalmic compositions, such as solutions, emulsions and suspensions and the like, are used in association with administering therapeutics or therapeutic components to or through the eyes. For example, an oil-in-water emulsion may be used as a carrier for a therapeutic component to be administered to the eyes. Such compositions often benefit from being effectively preserved, for example, using preservatives and/or concentrations of preservatives which do not cause significant detrimental effect to the composition or to the human or animal to whom the composition is administered.

There is a need for ophthalmic compositions which provide for an enhanced effect of a preservative component in the compositions thereby allowing for the use of preservatives and/or reduced concentrations of preservatives which do not cause such detrimental effects.

### SUMMARY OF THE INVENTION

New preserved ophthalmic compositions and methods of preserving ophthalmic compositions have been discovered. The present invention provides ophthalmic compositions which are effectively preserved, and preferably which have enhanced preservative efficacy. The present invention advantageously provides for compositions which allow for preservative components and/or reduced concentrations of preservative component which have reduced detrimental effects, e.g., side effects, to be employed.

In one broad aspect, the present invention provides for ophthalmic compositions comprising a carrier component, preferably comprising an aqueous component, a therapeutic component in a therapeutically effective amount, an oxy-chloro component in an amount effective in preserving the composition, and a borate component in an amount effective to enhance the preservative efficacy of the composition. In one embodiment, the preservative efficacy of the composition or of the oxy-chloro component is enhanced relative to a substantially identical composition without the borate component. Preferably, the composition includes a glycerin component in an amount effective to further enhance the preservative efficacy of the composition. The invention also provides for methods of enhancing an effect, preferably the preservative efficacy, of the oxy-chloro component in an ophthalmic composition. Such methods may include combining an amount of a borate component effective to enhance an effect of the oxy-chloro component with an ophthalmic composition which includes the oxy-chloro component.

In one embodiment, the ophthalmic composition includes an aqueous component and an oily component, and is, advantageously, in the form of an oil-in-water emulsion.

In one embodiment, the therapeutic component is a quinoxaline component. The quinoxaline component may include, for example, without limitation, quinoxalines, such as (2-imidozolin-2-ylamino) quinoxalines, salts thereof, such as ophthalmically acceptable acid additive salts thereof, and mixtures thereof.

In one embodiment, the quinoxaline component has the formula:

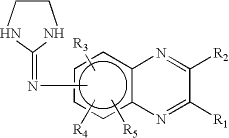

and ophthalmically acceptable acid addition salts thereof and mixtures thereof. The R₁ may be H, alkyl radicals containing 1 to 4 carbon atoms or alkoxy radicals containing 1 to 4 carbon atoms, R₂ may be H, alkyl radicals containing 1 to 4 carbon atoms, for example, methyl radicals or alkoxy radicals containing 1 to 4 carbon atoms. The 2-imidazolin-2-ylamino group may be in any of the 5-, 6-, 7-, or 8-positions of the quinoxaline nucleus. The R₃, R₄ and R₅ each may be located in any one of the remaining 5-, 6-, 7-, or 8-positions of the quinoxaline nucleus. Each of the 5-, 6-, 7-, and 8- may be Cl, Br, H or alkyl radicals containing 1 to 3 carbon atoms, for example, a methyl radical. For example, without limitation, the quinoxaline may have one of the following formulas:

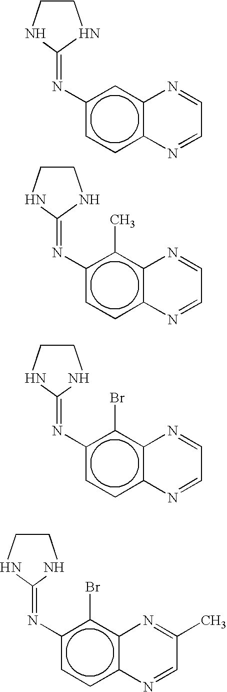

A very useful quinoxaline component, includes one or more chosen from 5-bromo-6-(2-imidozolin-2-ylamino) quinoxalines, salts thereof and mixtures thereof.

In one embodiment, the oxy-chloro component comprises a chlorite component. The oxy-chloro component may be a stabilized chlorine dioxide or a stabilized oxy-chloro complex.

The oxy-chloro component may be present in an amount in a range of about 1 ppm to about 5000 ppm, for example, about 10 ppm to about 1000 ppm or about 20 ppm to about 500 ppm of the composition. Advantageously, the oxy-chloro component is present in an amount greater than about 75 ppm, for example, in a range of greater than about 75 ppm to about 500 ppm or about 1000 ppm or about 5000 ppm.

In one embodiment, the borate component may include without limitation, boric acid, salts thereof, for example, borates, and mixtures thereof. The borate component may be present in amount in a range of about 0.01% to about 10%, for example, about 0.05% or about 0.1% to about 2% or about 5% (w/v) of the composition.

In one embodiment, the borate component is present in an amount between about 0.01% and about 3%.

In one embodiment, the presence of mannitol in the present compositions has been found to have a detrimental effect on the preservative efficacy of the compositions. In one particularly useful embodiment, the present compositions include substantially no mannitol and the like materials, to substantially eliminate this detrimental effect.

In one embodiment, the present compositions include a glycerin component in an amount effective to further enhance preservative efficacy. The glycerin component may be present in an amount effective to enhance the preservative efficacy of the oxy-chloro component or the combination of the oxy-chloro component and the borate component. Advantageously, the preservative efficacy of the oxy-chloro component or present composition including a glycerin component is enhanced relative to a substantially identical composition without the glycerin component. The glycerin component may be present in a composition that includes a borate component. In addition, the glycerin component may be present in a composition that does not include a borate component.

The present invention also provides for ophthalmic compositions which include an aqueous component, an oily component and a therapeutic component, such as a quinoxaline component. In one embodiment, the pH of the aqueous component is effective, and may be controlled or adjusted, to produce a desired partitioning of the therapeutic component, e.g., the quinoxaline component, for example, such that a major portion, that is about 50% or more, preferably more than about 50%, of the therapeutic component (quinoxaline component) is located in the aqueous component, for example, an aqueous phase, or in the oily component, for example, an oily phase.

The invention also provides for methods for producing a desired partitioning of a therapeutic component (quinoxaline component) in an ophthalmic composition. The method may include setting or adjusting the pH of an aqueous component of a composition comprising an aqueous component and a non-aqueous component at or to desired value.

The pH of a composition may be between about 3.0 and about 9.0, for example, between about 4.0 or about 5.0 to about 7.5 or about 8.5, or between about 7.5 and about 8.0 e.g., about 7.9.

In one embodiment, the therapeutic component is partitioned such that more than about 50% of the component is located in the aqueous component. For example, the therapeutic component may be partitioned such that more than about 60% or more than about 70% or more than about 80% or more than about 90% of the therapeutic component is located in the aqueous component.

Alternatively, the therapeutic component may be partitioned such that more than about 50% of the component is located in the oily component. For example, the therapeutic component may be partitioned more than about 60% or more than about 70% or more than about 80% or more than about 90% of the therapeutic component is located in the oily component.

In one useful embodiment, the present ophthalmic compositions comprise an aqueous component, a polyanionic component present in an amount effective to provide lubrication to an eye when the composition is administered to an eye, an oxy-chloro component present at a concentration of greater than about 75 ppm and a borate component present in an amount effective to enhance a preservative efficacy of the composition relative to a substantially identical composition without the borate component.

Each and every feature described herein, and each and every combination of two or more of such features, is included within the scope of the present invention provided that the features included in such a combination are not mutually inconsistent.

These and other aspects and advantages of the present invention are apparent in the following detailed description, examples and claims.

### DETAILED DESCRIPTION

The present invention relates to preserved ophthalmic compositions, for example, such compositions in which the effect or efficacy of the preservative is enhanced. The present invention also relates to controlling or adjusting the partitioning of a therapeutic component in an ophthalmic composition between an aqueous component of the composition and a non-aqueous component, for example, an oily phase, of the composition. In one embodiment, the compositions are ophthalmic compositions useful for drug delivery to or through the eye, for eye drops to treat dry eye, for otherwise caring for the eye and for caring for contact lenses, which compositions are benefited from being preserved. In certain embodiments, the present compositions can be, for example, artificial tear compositions, eyewash compositions, irrigating compositions for use during eye surgery and the like.

The compositions of the present invention are advantageously ophthalmically acceptable, for example, are substantially non-toxic and/or non-irritating and/or non-damaging to the eye, and can provide at least one benefit to the human or animal to whom the composition is administered, for example, can provide medication to the human or animal, can provide a protective function for ocular cells and tissues, and the like.

The present invention provides for the inclusion of an oxy-chloro-containing preservative component. A preservative component may include one or more preservatives. Useful preservatives include those that may derive their antimicrobial activity through a chemical or physiochemical interaction with the microbes or microorganisms.

Preferably, the oxy-chloro component is present in a carrier component, for example, an aqueous carrier component, such as a liquid aqueous medium, at an ophthalmically acceptable or safe concentration.

The concentration of oxy-chloro component selected depends, for example, on the effectiveness of the specific oxy-chloro component in preventing growth, or the killing, of bacteria, fungi, and/or protozoa in a preserved composition.

Very useful oxy-chloro components useful as preservatives in accordance with the present invention include hypochlorite components, for example, hypochlorites; chlorate components, for example chlorates; perchlorate components, for example perchlorates; and chlorite components, for example, chlorites.

Particularly useful oxy-chloro components include chlorite components. Examples of chlorite components include, without limitation, stabilized chlorine dioxide (SCD), metal chlorites, such as alkali metal and alkaline earth metal chlorites, and the like and mixtures thereof. Technical grade sodium chlorite is a very useful oxy-chloro component. The exact chemical composition of many chlorite components, for example, SCD, is not completely understood. The manufacture or production of certain chlorite components is described in McNicholas U.S. Pat. No. 3,278,447, which is incorporated in its entirety herein by reference. Specific examples of useful SCD products include that sold under the trademark Dura Klor by Rio Linda Chemical Company, Inc., and that sold under the trademark Anthium Dioxide by International Dioxide, Inc.

In another broad aspect of the present invention, an oxy-chloro component is present in a composition in an effective amount to at least aid in preserving, for example, in an amount effective to preserve, one or more components of the composition. Preferably, the oxy-chloro component is such so as to not substantially or significantly detrimentally affect the functioning of other components in the compositions, such as for example, a therapeutic component, e.g., a quinoxaline component, included in the composition.

In one embodiment, the oxy-chloro component is employed in a concentration of about 0.01 ppm or more. For example, the oxy-chloro may be employed in an amount in a range of about 0.1 ppm to about 4000 ppm or about 5000 ppm. In another example, the oxy-chloro may be employed in an amount in a range of about 0.1 ppm to about 2000 ppm or about 3000 ppm. In another example, the oxy-chloro may be employed in an amount in a range of about 0.1 ppm or 1.0 ppm to about 500 ppm or about 1000 ppm. In one embodiment, the oxy-chloro is present in an amount in a range of about 1.0 ppm to about 500 ppm.

Very effective concentrations of oxy-chloro components in the present compositions are greater than about 75 ppm. Such concentrations very effectively preserve the compositions without detrimentally effecting the other components of the compositions and without causing significant detrimental effects to the human or animal to whom the composition is administered. Such concentrations of oxy-chloro component, together with a borate component and/or a glycerin component, described elsewhere herein, provide for enhanced preservative efficacy and provide for acceptably long product shelf life. Advantageously, the oxy-chloro component is present in an amount in a range of greater than about 75 ppm to about 2000 ppm or about 3000 ppm or about 5000 ppm.

One important feature of the present invention is the inclusion of a borate component in the present compositions. A borate component is shown to be effective to enhance the effect of the oxy-chloro component in the present ophthalmic compositions. For example, the borate component may enhance the antibacterial and/or antifungal activity of the oxy-chloro component in the ophthalmic compositions. In one embodiment, the borate component prolongs the shelf life of a composition relative to a substantially identical composition without the borate component. The presently useful borate components include, without limitation, boric acid, salts of boric acid, and the like and mixtures thereof. Examples include, without limitation, borax, sodium tetraborate, sodium perborate, orthoboric acid, metaboric acid, mixtures thereof and the like. In addition, the present invention contemplates the use of any suitable boron-containing compound, for example, a boron-containing compound which is ophthalmically acceptable in the present compositions, which is effective to enhance the preservative efficacy of a composition in accordance with the present invention.

A borate component may be present in a composition in any amount which may be effective to enhance the effect of the oxy-chloro component in the composition. In one embodiment, the borate component is employed in a composition in concentration of about 0.001% (w/v) or more. For example, the borate component may be employed in an amount in a range of about 0.001% to about 10% (w/v) or about 20% (w/v). In another example, the borate component may be employed in an amount in a range of about 0.005% to about 5% (w/v) or about 10% (w/v). In another example, the borate component may be employed in an amount in a range of about 0.005% or 0.01% to about 2% (w/v) or about 4% (w/v). Advantageously, the borate component is present in an amount in a range of about 0.01% to about 1% (w/v).

In another important aspect of the present invention, a glycerin component, such as, without limitation, glycerin and the like and mixtures thereof, can enhance an effect of the oxy-chloro component in a composition. For example, a glycerin component can enhance an effect of the oxy-chloro component in a composition when the composition also includes a borate component. The glycerin component may be present in a composition in any amount effective to enhance the effect of the oxy-chloro component. For example, the glycerin component may enhance the antibacterial and/or antifungal activity of the oxy-chloro component in a composition. In one embodiment, the glycerin component prolongs the shelf life of a composition relative to a substantially identical composition without the glycerin component. Glycerin components are very useful to enhance the preservative efficacy of ophthalmic compositions comprising emulsions having aqueous components and oily components.

In one embodiment, the glycerin component is employed in a composition in concentration of about 0.001% (w/v) or more. For example, the glycerin component may be employed in an amount in a range of about 0.001% to about 30% (w/v). The glycerin component may be employed in an amount in a range of about 0.005% or about 0.01% or about 0.1% to about 10% (w/v) or about 15% (w/v) or about 20% (w/v) or about 30% (w/v). Preferably, the glycerin component is present in an amount in a range of about 0.1% to about 5% (w/v).

In a further important aspect of the present invention, the present compositions are substantially free of certain carbohydrates and/or alcohols or sugar-alcohols (i.e., polyols). For example, a composition may be substantially free of mannitol, sorbitol, xylitol and the like and mixtures thereof. In one embodiment, the oxy-chloro component is included in a composition that is substantially free of one or more certain carbohydrates, alcohols and/or polyols, as described elsewhere herein, and has one or more enhanced effects, preferably enhanced preservative efficacy, relative to a substantially identical composition which includes such substances, for example, which includes 1.5% (w/v) of one or more such carbohydrates, alcohols and/or polyols. In one particularly useful embodiment, a composition is substantially free of mannitol.

In one embodiment, the present compositions which are substantially free of mannitol have enhanced preservative efficacy relative to a substantially identical composition which includes 1.5% (w/v) of mannitol. In one embodiment, the preserved composition substantially free of mannitol has prolonged shelf life relative to a substantially identical composition which includes 1.5% (w/v) of mannitol.

A therapeutic component may be included in compositions of the present invention. Examples of useful therapeutic components include, but are not limited to, NMDA antagonists; antibacterial substances such as beta-lactam antibiotics, for example, cefoxitin, n-formamidoylthienamycin and other thienamycin derivatives, tetracyclines, chloramphenicol, neomycin, carbenicillin, colistin, penicillin G, polymyxin B, vancomycin, cefazolin, cephaloridine, chibrorifamycin, gramicidin, bacitracin and sulfonamides; aminoglycoside antibiotics such as gentamycin, kanamycin, amikacin, sisomicin and tobramycin; quinolones such as norfloxacin, ofloxacin and the like; nitrofurazones and analogs thereof; antihistaminics and decongestants such as pyrilamine, chlorpheniramine, tetrahydrazoline, antazoline and analogs thereof; mast-cell inhibitors of histamine release such as cromolyn and the like; anti-inflammatories such as cortisone, hydrocortisone, hydrocortisone esters, betamethasone, dexamethasone, dexamethasone sodium phosphate, prednisone, methylprednisolone, medrysone, fluorometholone, prednisolone, prednisolone sodium phosphate, triamcinolone, indainethacin, sulindac, and analogs thereof; miotics and anticholinergics such as echothiophate, pilocarpine, physostigmine salicylate, diisopropylfluorophosphate, epinephrine, dipivaloylepinephrine, neostigmine echothiopate iodide, demecarim bromide, carbamoyl choline chloride, methacholine, bethanechol and analogs thereof; mydriatics such as atrophine, homatropine, scopolamine, hydroxyamphetamine, ephedrine, cocaine, tropicamide, phenylephrine, cyclopentolate, oxyphenonium, eucatropine; and the like and mixtures thereof.

Other therapeutic components include, without limitation: antiglaucama drugs, for example, timolol, and especially its maleic salt and R-timolol, and combinations of timolol, timolol maleate and/or R-timolol with pilocarpine; adrenergic agonists and/or antagonists such as epinephrine and epinephrine complexes, and prodrugs such as bitartrate, borate, hydrochloride and dipivefrine derivatives; carbonic anhydrase inhibitors such as acetazolamide, dichlorphenamide, 2-(p-hydroxyphenyl)-thiothiophene-sulfonamide, 6-hydroxy-2-benzothiazolesulfonamide, and 6-pivaloyloxy-2-benzothiazolesulfonamide; antiparasitic compounds and/or anti-protozoal compounds such as ivermectin, pyrimethamine, trisulfapidimidine, clindamycin and corticosteroid preparations; compounds having antiviral activity such as acyclovir, 5-iodo-2′-deoxyuridine (IDU), adenosine arabinoside (Ara-A), trifluorothymidine, interferon, and interferon-inducing agents such as poly I:C; antifungal agents such as amphotericin B, nystatin, flucytosine, natamycin and miconazole; anesthetic agents such as etidocaine cocaine, benoxinate, dibucaine hydrochloride, dyclonine hydrochloride, naepaine, phenacaine hydrochloride, piperocaine, proparacaine hydrochloride, tetracaine hydrochloride, hexylcaine, bupivacaine, lidocaine, mepivacaine and prilocaine; ophthalmic diagnostic agents, such as: (a) those used to examine the retina, for example, sodium fluorescein, (b) those used to examine the conjunctiva, cornea and lacrimal apparatus, for example, fluorescein and rose bengal and (c) those used to examine abnormal pupillary responses, for example, methacholine, cocaine, adrenaline, atropine, hydroxyamphetamine and pilocarpine; ophthalmic agents used as adjuncts in surgery, for example, alpha-chymotrypsin and hyaluronidase; chelating agents, for example, ethylenediaminetetraacetic acid (EDTA), salts thereof, and deferoxamine; immunosuppressants and anti-metabolites, for example, methotrexate, cyclophosphamide, 6-mercaptopurine and azathioprine; and combinations of the agents mentioned above, such as antibiotics/antiinflammatories combinations, for example, the combination of neomycin sulfate and dexamethasone sodium phosphate, and combinations concomitantly used for treating glaucoma, for example, a combination of timolol maleate and aceclidine; and the like and mixtures thereof.

Other useful therapeutic components include ocular hypotensive agents such as disclosed in Woodward et al U.S. Pat. No. 5,688,819; pyranoquinolinone derivatives such as disclosed in Cairns et al U.S. Pat. No. 4,474,787; compounds having retinoid-like activities such as disclosed in Chandraratna U.S. Pat. No. 5,089,509; ketorolac/pyrrole-1-carboxylic acids such as disclosed in Muchowski et al U.S. Pat. No. 4,089,969; ofloxacins/benzoxazine derivatives such as disclosed in Hayakawa et al U.S. Pat. No. 4,382,892 and memantines such as disclosed in Lipton et al U.S. Pat. No. 5,922,773. The disclosure of each of U.S. Pat. Nos. 5,688,819; 4,474,787; 5,089,509; 4,089,969; 4,382,892; and 5,922,773 is incorporated herein in its entirety by reference.

In one useful embodiment, the present therapeutic components include adrenergic agonists. The adrenergic agonists may be amine-containing chemical entities with pKa's of greater than about 7, for example, in a range of about 7 (or greater than about 7) to about 9.

In one embodiment, the useful therapeutic components include alpha-adrenergic agonists. Examples of alpha-adrengergic agonists include, but are not limited to, adrafinil, adrenolone, amidephrine, apraclonidine, budralazine, quinoxalines, clonidine, cyclopentamine, detomidine, dimetofrine, dipivefrin, ephedrine, epinephrine, fenoxazoline, guanabenz, guanfacine, hydroxyamphetamine, ibopamine, indanazoline, isometheptene, mephentermine, metaraminol, methoxamine, methylhexaneamine, metizolene, midodrine, naphazoline, norepinephrine, norfenefrine, octodrine, octopamine, oxymetazoline, phenylephrine, phenylpropanolamine, phenylpropylmethylamine, pholedrine, propylhexedrine, pseudoephedrine, rilmenidine, synephrine, tetrahydrozoline, tiamenidine, tramazoline, tuaminoheptane, tymazoline, tyramine, xylometazoline, and the like and mixtures thereof.

In one useful embodiment, the therapeutic components include alpha-2-adrenergic agonists. As used herein, the term “alpha-2 adrenergic agonist” includes chemical entities, such as compounds, ions, complexes and the like, that may produce a net sympatholytic response, resulting in increased accommodation, for example, by binding to presynaptic alpha-2 receptors on sympathetic postganglionic nerve endings or, for example, to postsynaptic alpha-2 receptors on smooth muscle cells. A sympatholytic response is characterized by the inhibition, diminishment, or prevention of the effects of impulses conveyed by the sympathetic nervous system. The alpha-2 adrenergic agonists of the invention may bind to the alpha-2 adrenergic receptors presynaptically, causing negative feedback to decrease the release of neuronal norepinephrine. Additionally, they also may work on alpha-2 adrenergic receptors postsynaptically, inhibiting beta-adrenergic receptor-stimulated formation of cyclic AMP, which contributes to the relaxation of the ciliary muscle, in addition to the effects of postsynaptic alpha-2 adrenergic receptors on other intracellular pathways. Activity at either pre- or postsynaptic alpha-2 adrenergic receptors may result in a decreased adrenergic influence. Decreased adrenergic influence results in increased contraction resulting from cholinergic innervations. Alpha-2 adrenergic agonists also include compounds that have neuroprotective activity. For example, 5-bromo-6-(2-imidozolin-2-ylamino) quinoxaline is an alpha-2-adrenergic agonist which has a neuroprotective activity through an unknown mechanism.

Without limiting the invention to the specific groups and compounds listed, the following is a list of representative alpha-2 adrenergic agonists useful in this invention: imino-imidazolines, including clonidine, apraclonidine; imidazolines, including naphazoline, xymetazoline, tetrahydrozoline, and tramazoline; imidazoles, including detomidine, medetomidine, and dexmedetomidine; azepines, including B-HT 920 (6-allyl-2-amino-5,6,7,8 tetrahydro-4H-thiazolo[4,5-d]-azepine and B-HT 933; thiazines, including xylazine; oxazolines, including rilmenidine; guanidines, including guanabenz and guanfacine; catecholamines and the like.

Particularly useful alpha-2-adrenergic agonists include quinoxaline components. In one embodiment, the quinoxaline components include quinoxalines, derivatives thereof and mixtures thereof. The derivatives of quinoxaline include, without limitation, (2-imidozolin-2-ylamino) quinoxalines, salts thereof and mixtures thereof. In one embodiment, the derivatives of quinoxaline include 5-halide-6-(2-imidozolin-2-ylamino) quinoxalines, salts thereof and mixtures thereof. The “halide” of the 5-halide-6-(2-imidozolin-2-ylamino) quinoxalines may be a fluorine, a chlorine, an iodine, or preferably, a bromine, to form 5-bromo-6-(2-imidozolin-2-ylamino) quinoxaline (brimonidine), also known as brimonidine.

Other useful quinoxalines and quinoxaline derivatives are well known. For example, useful quinoxalines and derivatives of a quinoxaline include the ones disclosed by U.S. Pat. No. 5,021,416; U.S. Pat. No. 5,703,077; and U.S. Pat. No. 3,890,319. The disclosure of each of these three patents is incorporated in its entirety by reference herein.

The quinoxaline and derivatives thereof, for example, brimonidine, are amine-containing and preferably have pKa's of greater than about 7, preferably about 7.5 to about 9.

Analogs, salts, for example, ophthalmically acceptable salts and other derivatives of the foregoing chemical entities that function in a similar manner to provide a desired therapeutic effect also are specifically contemplated for use as therapeutic components in the present compositions.

In one useful embodiment, the amount of therapeutic component in the present composition is in the range of about 0.01% to about 30% (w/v). The amount of therapeutic component may be in the range of about 0.1% (w/v) to about 10% (w/v). For example, the amount of therapeutic component may be in the range of about 0.1% (w/v) to about 0.6% (w/v). In one embodiment, the therapeutic component is an adrenergic agonist and is present in the composition in the range of about 0.1% (w/v) to about 0.6% (w/v), for example, about 0.15% (w/v).

The present compositions may conveniently be presented as solutions or suspensions in aqueous liquids or non-aqueous liquids, or as oil-in-water or water-in-oil liquid emulsions. The present compositions may include one or more ingredients which are conventionally employed in compositions of the same general type.

The present compositions in the form of aqueous suspensions may include excipients suitable for the manufacture of aqueous suspensions. Such excipients include without limitation, suspending agents, for example, sodium carboxymethylcellulose, methylcellulose, hydroxypropyl-methylcellulose, sodium alginate, polyvinylpyrrolidone, gun tragacanth and gun acacia; dispersing or wetting agents may be a naturally occurring phosphatide, for example, lecithin, or condensation products of ethylene oxide with long chain aliphatic alcohols, for example, heptadeca-ethyleneoxycetanol, or condensation products of ethylene oxide with partial esters derived from fatty acids and a hexitol such as polyoxyethylene sorbitol mono-oleate, or condensation products of ethylene oxide with partial esters derived from fatty acids and hexitol anhydrides, for example, polyoxyethylene sorbitan mono-oleate, and the like and mixtures thereof.

The present compositions in the form of oily suspensions may be formulated in a vegetable oil, for example, olive oil, castor oil, soy oil, sesame oil or coconut oil, or in a mineral oil such as liquid paraffin. Such suspensions may include a thickening agent, for example, beeswax, hard paraffin or cetyl alcohol.

The present compositions may be in the form of oil-in-water emulsions. The oily phase may be a vegetable oil, for example, castor oil, olive oil, soy oil, or arachis oil, or a mineral oil, for example, liquid paraffin, and the like and mixtures thereof. Suitable emulsifying agents may be naturally-occurring gums, for example, gum acacia or gum tragacanth, naturally-occurring phosphatides, for example, soya bean lecithin, and esters or partial esters derived from fatty acids and hexitol anhydrides, for example, sorbitan mono-oleate, and condensation products of the said partial esters with ethylene oxide, for example, polyoxyethylene sorbitan mono-oleate.

Very useful oil/water emulsions which can be employed in the present invention are described in commonly assigned U.S. patent application Ser. No. 10/349,466, filed Jan. 22, 2003. The disclosure of the above-noted application is hereby incorporated in its entirety herein by reference.

In a useful embodiment, the present invention provides for the partitioning of a therapeutic component in a composition. In this embodiment, a composition includes two or more phases into which a therapeutic component can be partitioned. For example, the composition may include an aqueous phase component and a non-aqueous phase component such as an oily phase, and be present in the form of an oil-in-water emulsion or a water-in-oil liquid emulsion. Partitioning of a therapeutic component in a composition means that a certain fraction (or percent) of the total therapeutic component is present in one or more phases of the composition. For example, a first fraction of the therapeutic component may be present in one phase of a composition (e.g., an aqueous component or a non-aqueous component, for example, an oily component) and the remainder of the therapeutic component is present in the other phase of the composition.

In one important aspect of the present invention, the partitioning of the one or more therapeutics may be determined by the pH of an aqueous phase component of a composition.

Without wishing to limit the present invention to any theory of operation, it is believed that a therapeutic component can be present as either a charged species or as a free base or a combination thereof. For example, a large percentage of certain therapeutic components (e.g., brimonidine) is present as a free base at a pH near 8.0, for example, at pH 7.9. It is believed that a free base of a therapeutic component is more hydrophobic than a charged species of the therapeutic component, and therefore, the free base more readily partitions into the oily component of a composition than the charged species of the therapeutic component. In addition, it is believed that certain therapeutic components which are hydrophobic can more readily penetrate the cornea of an eye relative to less hydrophobic therapeutic components.

In one embodiment, a greater fraction of the therapeutic component may be advantageously present in the oily component than in the aqueous component. In another embodiment, a greater fraction of the therapeutic component may be advantageously present in the aqueous component than in the oily component. In another embodiment, substantially equal fractions of the therapeutic component may be advantageously present in the aqueous component and in the oily component. In one useful embodiment, the therapeutic component (e.g., brimonidine) may be present in an ophthalmic composition including an aqueous component which has a pH of about 7.9 in which the therapeutic component may be present as a free base in a larger percentage, relative to a substantially identical composition with a lower pH.

The pH of a composition may be such that the therapeutic component may be present in a particular phase of a composition in any percentage of the total therapeutic component present in the composition. For example, the pH may be in a range of about pH 5.0 to about pH 10.0 or about pH 5.5 to about pH 9.5 or about pH 6.0 to about pH 9.0 or about pH 6.5 to about ph 8.5 or about pH 7.0 to about pH 8.0.

In one embodiment, a first therapeutic component present in a composition may be partitioned to one phase of the composition while a second therapeutic component present in the composition may be partitioned to another phase of the composition.

For example, without wishing to limit the invention to any theory of operation, it is believed that in certain oil-in-water emulsion compositions or water-in-oil emulsion compositions, that at a pH value of about 6.5, greater than about 90% (e.g., greater than about 99%) of a therapeutic component, for example brimonidine is partitioned in an aqueous component or aqueous phase of a composition. Similar compositions, at a pH value of about 8.0 have about 50% of the therapeutic component, for example, brimonidine, partitioned in the oily phase or oily component of the composition.

The carrier component of the present compositions is ophthalmically acceptable. A carrier component or other material is “ophthalmically acceptable” when it is substantially compatible with ocular tissue. That is, it does not cause significant or undue detrimental effects when brought into contact with ocular tissue. Preferably, the ophthalmically acceptable material is also substantially compatible with other components of the present compositions. The carrier component may include one or more components which are effective in providing such ophthalmic acceptability and/or otherwise benefiting the composition and/or the eye to which the composition is administered and/or the patient whose eye is being treated. Advantageously, the carrier component is aqueous-based, for example, comprising a major amount, that is at least about 50% by weight, of water.

Examples of suitable materials useful in the present carrier components include water, mixtures of water and water-miscible solvents such as lower alkanols or aralkanols, oily components, vegetable oils, polyalkylene glycols, petroleum-based jelly, ethyl cellulose, ethyl oleate, polyvinylpyrrolidone, isopropyl mirstate, other conventionally employed ophthalmically acceptable materials and the like and mixtures thereof.

The carrier component may also include auxiliary substances such as emulsifiers, wetting agents, bodying agents, buffer components, acids and/or bases, tonicity adjuster components, surfactant components, viscosity agents, lubricity components, preservative components, other materials useful in ophthalmic formulations and the like, including, but not limited to, such substances which are conventionally used in ophthalmic compositions.

Examples of optionally useful bodying agents include, but are not limited to, various polyethylene glycols, carbowaxes, petroleum jelly and the like.

Suitable buffers include, but are not limited to, inorganic buffers such as phosphate buffers, borate buffers and the like, and organic buffers, such as acetate buffers, citrate buffers, tromethamine and the like.

Tonicity adjusters optionally useful in the present compositions include, but are not limited to, dextrose, potassium chloride and/or sodium chloride and the like, preferably sodium chloride.

Acids optionally useful in the present compositions include boric acid, hydrochloric acid, acetic acid, other acids which are ophthalmically acceptable in the concentrations used, and the like.

Bases which may be included in the present compositions include, but are not limited to, sodium and/or potassium hydroxides, other alkali and/or alkaline earth metal hydroxides, organic bases, other bases which are ophthalmically acceptable in the concentrations used, and the like.

The acid/bases/buffers preferably are included, if at all, to provide and/or maintain the present compositions at a pH in the physiologically acceptable range, more preferably in a range of about 4 to about 8.5, still more preferably about 6 to about 8, and especially about 6.8 to about 8.

Surfactant components optionally useful in the compositions of the present invention include, but are not limited to, lipoprotein detergents that when present in the compositions reduce the surface tension between the compositions and the eye (lacrimal) fluid. Preferably, nonionic surfactants are used.

Viscosity agents optionally useful in the compositions of the present invention include, but are not limited to, cellulose derivatives such as hydroxypropylmethyl cellulose, carboxymethyl cellulose, hydroxyethyl cellulose, other viscosity inducing materials useful in ophthalmic formulations, and the like.

In one very useful embodiment, the present compositions include a polyanionic component. Advantageously, the polyanionic component is present in an amount effective to provide lubrication to an eye when the composition is administered to the eye. The polyanionic component is often present in an amount of at least about 0.1% w/v of the composition. For example, the polyanionic component may be present in an amount in a range of about 0.1% or about 0.2% to about 1% (w/v) or 5% (w/v) or about 10% (w/v) of the composition. In another example, the polyanionic component is present in an amount in a range of about 0.6% to about 1.8% (w/v) of the composition.

Any suitable polyanionic component can be employed in accordance with the present invention provided that it functions as described herein and has no substantial detrimental effect on the composition as a whole or on the eye to which the composition is administered. Such polyanionic component should be ophthalmically acceptable, compatible with the other components of the composition, and effective, in ophthalmically reasonable concentrations, to facilitate administration of a therapeutic component, for example, a quinoxaline component to the eye when administered to the eye and to otherwise function in accordance with the present invention.

As used herein, the term “polyanionic component” refers to a chemical entity, for example, an ionically charged species, such as an ionically charged polymeric material, which includes more than one discrete anionic charge, that is multiple discrete anionic charges. Preferably, the polyanionic component is selected from the group consisting of polymeric materials having multiple anionic charges and mixtures thereof.

Examples of suitable polyanionic components useful in the present compositions include, without limitation, anionic cellulose derivatives, anionic acrylic acid-containing polymers, anionic methacrylic acid-containing polymers, anionic amino acid-containing polymers and the like and mixtures thereof. Anionic cellulose derivatives are particularly useful in the present invention.

A useful class of polyanionic components are one or more polymeric materials having multiple anionic charges. Examples include, but are not limited to:

metal carboxy methylcelluloses

metal carboxy methylhydroxyethylcelluloses

metal carboxy methylstarchs

metal carboxy methylhydroxyethylstarchs

ammonium methylcelluloses

amino compound methylcelluloses

hydrolyzed polyacrylamides and polyacrylonitriles

heparin

gucoaminoglycans

hyaluronic acid

chondroitin sulfate

dermatan sulfate

peptides and polypeptides

alginic acid

metal alginates

homopolymers and copolymers of one or more of:

acrylic and methacrylic acids

metal acrylates and methacrylates

vinylsulfonic acid

metal vinylsulfonate

amino acids, such as aspartic acid,

glutamic acid and the like

metal salts of amino acids

p-styrenesulfonic acid

metal p-styrenesulfonate

2-methacryloyloxyethylsulfonic acids

metal 2-methacryloyloxethylsulfonates

3-methacryloyloxy-2-hydroxypropylsulonic acids

metal 3-methacryloyloxy-2-hydroxypropylsulfonates

2-acrylamido-2-methylpropanesulfonic acids

metal 2-acrylamido-2-methylpropanesulfonates

allylsulfonic acid

metal allylsulfonate and the like.

Excellent results may be achieved using polyanionic components selected from carboxy methylcelluloses and mixtures thereof, for example, alkali metal and/or alkaline earth metal carboxy methylcelluloses.

In one embodiment, the polyanionic component includes more than one polyanionic component portion, each polyanionic component portion having a molecular weight different from another polyanionic component portion comprising the polyanionic component.

In one very useful embodiment, the polyanionic component includes a first polyanionic component portion having a first molecular weight, and a second polyanionic component having a second molecular weight.

In one embodiment, each of the polyanionic component portions is present in an amount of at least about 0.01% w/v of the composition. For example, each of the polyanionic component portions may be present in an amount in a range of about 0.01% or about 0.1% or about 0.2% or about 2% (w/v) or about 5% (w/v) or about 10% (w/v) of the composition. In another example, each of the polyanionic component portions is present in an amount in a range of about 0.1% to about 2.0% (w/v) of the composition.

As noted above, each of the polyanionic component portions may have a different molecular weight. In one embodiment, the first polyanionic component portion has a first molecular weight which is greater than the second molecular weight of the second polyanionic component portion. The difference in molecular weight between the polyanionic component portions, for example, between the first and second polyanionic component portions, may be at least about 10,000, for example, at least about 50,000.

In one embodiment, the weight ratio of the first polyanionic component portion and the second polyanionic component portion is in a range of about 0.02 to about 50. For example, the weight ratio of the first polyanionic component portion and the second polyanionic component portion is in a range of about 0.25 to about 4.

As used herein the term “molecular weight” refers to weight average molecular weight, as that term is commonly known within the polymer art, and can be measured or determined using procedures and/or techniques well known in this art.

In one useful embodiment, at least one of the polyanionic component portions are selected from anionic cellulosic derivatives and mixtures thereof. A very useful embodiment provides that all of the polyanionic component portions be selected from the group consisting of carboxy methyl celluloses and mixtures thereof.

Other suitable polyanionic components may be employed. For example, at least one, for example all, of the polyanionic component portions may be selected from anionic homopolymers and copolymers comprising units of one or more of acrylic acid, methacrylic acid, metal acrylates and metal methacrylates, and mixtures thereof. A very useful polyanionic component from which at least one of the first and second polyanionic component portions may be selected are homopolymers and copolymers comprising units of one or more of acrylic acid, metal acrylates and mixtures thereof. The polyanionic component can include three (3) or more anionic (or negative) charges. In the event that the polyanionic component is a polymeric material, it is preferred that each of the repeating units of the polymeric material include a discrete anionic charge. Particularly useful anionic components are those which are water soluble, for example, soluble at the concentrations used in the present compositions at ambient (room) temperature.

Each polyanionic component portion can have a different molecular weight. In one very useful embodiment, the polyanionic component includes a first polyanionic component portion having a first molecular weight; and a second polyanionic component having a second molecular weight. Advantageously, each of the polyanionic component portions is present in an amount effective to facilitate administration of a therapeutic component, for example, a quinoxaline component into the eye through the cornea of the eye when the composition is administered to the eye. Each of the polyanionic component portions can be present in an amount of at least about 0.1% w/v of the composition.

In one embodiment, the at least two polyanionic component portions, for example, the first and second polyanionic component portions, other than having different molecular weights, have substantially similar chemical structures. However, the at least two polyanionic component portions can have different chemical structures.

Each of the polyanionic component portions, for example, the first and second polyanionic component portions, can be separately derived. In other words, each of the polyanionic component portions can be combined into the present compositions as separate materials.

The present compositions preferably have viscosities in excess of the viscosity of water. In one embodiment, the viscosity of the present compositions is at least about 15 cps (centipoise), for example, in a range of about 15 cps to about 2000 cps or about 3,000 cps. Advantageously, the viscosity of the present composition may be in a range of about 30 cps or about 70 cps to about 750 cps or about 1000 cps. In one embodiment, the viscosity of a composition is a range of about 15 cps or about 50 cps to about 200 cps. In another embodiment, the viscosity of a composition is in a range of about 30 cps to about 5000 cps or about 200 cps to about 4000 cps. In still another embodiment, the viscosity of a composition is in a range of about 200 cps to about 2000 cps.

The viscosity may be measured at a shear rate of between 1 and 10 per second. The viscosity of the present compositions can be measured in any suitable manner. A conventional Brookfield viscometer can be used to measure such viscosities. The compositions can be either newtonian or non-newtonian compositions. Shear-thinning characteristics of non-neutonian compositions that result in the composition having a lower viscosity under conditions of physical shear (e.g., blinking) may allow for a higher initial viscosity for non-newtonian compositions than neutonian compositions.

As noted previously, each of the polyanionic component portions, that is, for example, at least the first and second polyanionic component portions, can be present in an amount of at least about 0.1% (w/v) of the composition. In one very useful embodiment, the polyanionic component is present in an amount in a range of about 0.2% to about 5%, for example, about 0.4% to about 2.5%, or for example, about 0.6% to about 1.8% or for example, about 0.8% to about 1.3% (w/v) of the composition.

The weight ratio of the first polyanionic component portion to the second polyanionic component portion may vary over a wide range. In one embodiment, the ratio weight of the first portion to the second portion is in the range of about 0.02 to about 50, preferably about 0.1 to about 10, and more preferably about 0.25 to about 4.

The different, for example, first and second, polyanionic component portions of the present compositions may be separately derived. Put another way, the different (e.g., first and second) polyanionic component portions can be blended into the present compositions from different sources. The molecular weights of the different polyanionic component portions can differ by at least about 10,000, for example, at least about 50,000.

In a one useful embodiment, the polyanionic component further comprises a third polyanionic component portion having a third molecular weight which is different from the first and second molecular weights. The third polyanionic component portion preferably is present in an amount effective to facilitate administration of a therapeutic component, for example, a brimonidine component, to an eye relative to a substantially identical composition with no third polymeric component portion.

Also included within the scope of this invention are preserved compounds which increase in viscosity upon administration to the eye. For example, “gelling polysaccharides” which are disclosed in U.S. Pat. No. 5,212,162 which is incorporated in its entirety herein by reference. Also disclosed in this patent are ophthalmic formulations containing carrageenans and furcellarans which are administered as partially gelled liquids which gel upon instillation into the eye. Additionally, U.S. Pat. Nos. 4,136,173, 4,136,177, and 4,136,178, disclose the use of therapeutic compositions containing xanthan gum and locust bean gum which are delivered in liquid form to the eye and which gel upon instillation. U.S. Pat. No. 4,861,760 discloses ophthalmological compositions containing gellan gum which are administered to the eye as non-gelled liquids and which gel upon instillation. The disclosure of each of these four patents is incorporated in its entirety herein by reference.

Also within the scope of this invention are preserved oils, ointments, gels and the like. The present compositions may include components, such as cyclodextrins, to enhance the solubility of one or more other components included in the compositions. For example, steroids, which are hydrophobic, often exhibit an increase in water solubility of one order of magnitude or more in the presence of cyclodextrins. Any suitable cyclodextrin component may be employed in accordance with the present invention. The useful cyclodextrin components include, but are not limited to, those materials which are effective in increasing the apparent solubility, preferably water solubility, of poorly soluble active components and/or enhance the stability of the active components and/or reduce unwanted side effects of the active components. Examples of useful cyclodextrin components include, but are not limited to: α-cyclodextrin, derivatives of α-cyclodextrin, β-cyclodextrin, derivatives of β-cyclodextrin, γ-cyclodextrin, derivatives of γ-cyclodextrin, carboxymethyl-β-cyclodextrin, carboxymethyl-ethyl-β-cyclodextrin, diethyl-β-cyclodextrin, dimethyl-β-cyclodextrin, methyl-β-cyclodextrin, random methyl-β-cyclodextrin, glucosyl-β-cyclodextrin, maltosyl-β-cyclodextrin, hydroxyethyl-β-cyclodextrin, hydroxypropyl-β-cyclodextrin, sulfobutylether-β-cyclodextrin, and the like and mixtures thereof. As used herein, the term “derivative”, as it relates to a cyclodextrin, means any substituted or otherwise modified compound which has the characteristic chemical structure of a cyclodextrin sufficiently to function as a cyclodextrin component, for example, to enhance the solubility and/or stability of active components and/or reduce unwanted side effects of the active components and/or to form inclusive complexes with active components, as described herein.

One or more additional components can be included in the present compositions based on the particular application for which the compositions are formulated. For example, the present compositions can be formulated to include a therapeutic component to be administered to the eyes.

The present preserved compositions may be administered to the eyes. These compositions, formulated appropriately, may be used in place of prior conventional compositions. For example, the compositions may be use in administering a therapeutic component to the eyes. In one embodiment, an antibiotic is administered to the eyes in a composition of the invention. In another example, the compositions of the invention may be used as a surgical irrigant.

The present compositions may also be used in the care of a contact lens, for example, to make wearing the lens safe and comfortable. The present compositions, formulated appropriately, may be used in conventional contact lens care regimens by using the present compositions in place of prior conventional compositions. In many instances, these contact lens care regimens involve contacting the lens with the present composition in an amount, and at conditions, effective to obtain the beneficial or desired contact lens care result.

The following non-limiting examples illustrate certain aspects of the present invention.

Each formulation set forth in the following examples is prepared by blending together the listed components in a conventional manner.

Each of these formulations is tested by performing an abbreviated preservative efficacy test using test organisms S. aureus, P. aeruginosa, c. albicans, E. coli and/or A. niger. The formulations are tested against United States Preservative Efficacy Test (USP), European Efficacy Test-A (EP-A) and European Efficacy Test-B (EP-B) criteria as indicated. Ten (10) ml of each formulation is challenged with approximately 10⁵ cfu/ml of test organism. At appropriate time intervals, the amount of bacterial and fungal survivors are assayed using Dey Engley broth (DE) as the neutralizer media. DE, along with filtration, is sufficient at neutralizing the antimicrobial agents in the compositions. One (1) ml of each sample is diluted into nine (9) ml of DE. One (1) ml of the 1:10 dilution is filtered through a 0.45 μm filter and washed with 100 ml of a saline/polysorbate 80 solution. After washing the filtrate a second time with 100 ml of saline/polysorbate 80 solution, the filtrate is placed onto a TSA plate for bacteria and SAB for fungi.

### EXAMPLE 1

The following four (4) formulations are prepared and tested. A summary of the test results is as follows:

| Components | Formulation 1 | Formulation 2 | Formulation 3 | Formulation 4 |
|---|---|---|---|---|
| Pemulen TR-2 (1) | 0.05 | | | |
| % (w/v) | | | | |
| Polysorbate 80 | 1 | | | |
| % (w/v) | | | | |
| Castor oil | 1.25 | | | |
| % (w/v) | | | | |
| Boric Acid | — | | | |
| % (w/v) | | | | |
| Mannitol | — | | | |
| % (w/v) | | | | |
| Glycerin | 2.2 | | | |
| % (w/v) | | | | |
| PH | 7.4 | | | |
| Purified Water | QS 100% | | | |
| Oxy-chloro | 0 | 25 | 50 | 150 |
| component (2) | | | | |
| (ppm) | | | | |
| USP | Fail | Pass | Pass | Pass |
| | P. aeruginosa | | | |
| | 0.6 log | | | |
| | reduction/7 d | | | |
| | E. coli 0.2 log | | | |
| | reduction/7 d | | | |
| EP-B | Fail | Fail | Fail | Pass |
| | S. aureus 0 log | C. albicans 0.2 log | C. albicans 0.4 log | |
| | reduction/24 hr | reduction/14 d | reduction/14 d | |
| | P. aeruginosa 0 log | A. niger 0.3 log | A. niger 0.4 log | |
| | reduction/24 hr | reduction/14 d | reduction/14 d | |
| | C. albicans 0 log | | | |
| | reduction/14 d | | | |
| | A. niger 0.3 log | | | |
| | reduction/14 d | | | |
| EP-A | Fail | Fail | Fail | Fail |
| | S. aureus 0 log/ | S. aureus 0.6 log | S. aureus 1.2 log | C. albicans 0.7 log |
| | 6 hr | reduction/6 hr | reduction/ | reduction/ |
| | P. aeruginosa | C. albicans 0.2 log | 6 hr | 7 d |
| | 0.3 log | reduction/ | C. albicans 0.4 | A. niger 0.8 log |
| | reduction/6 hr | 7 d | reduction/ | reduction/ |
| | C. albicans 0.2 log | A. niger 0.2 log | 7 d | 7 d |
| | reduction/7 d | reduction/7 d | A. niger 0.3 log | |
| | A. niger 0 log | | reduction/ | |
| | reduction/7 d | | 7 d | |

(1) C10 to C30 alkyl acrylate crosspolymer
(2) Stabilized chlorine dioxide, available from Allergan, Inc.

This example demonstrates that the use of an oxy-chloro component as a preservative, in certain instances, is not effective to meet certain preservative efficacy criteria, for example, European Efficacy Test-A (EP-A) or European Efficacy Test-B (EP-B). None of Formulations 1 to 4 include boric acid.

### EXAMPLE 2

Another formulation, Formulation 5, is prepared and tested. A summary of the test results, compared to Formulation 4, is as follows:

| Components | Formulation 4 | Formulation 5 |
|---|---|---|
| Oxy-chloro component | 150 | |
| (2) (ppm) | | |
| Pemulen TR-2 (1) | 0.05 | |
| % (w/v) | | |
| Polysorbate 80 | 1 | |
| % (w/v) | | |
| Castor oil | 1.25 | |
| % (w/v) | | |
| Purified Water | QS 100% | |
| Boric Acid | — | 0.15 |
| % (w/v) | | |
| Mannitol | — | 1.5 |
| % (w/v) | | |
| Glycerin | 2.2 | 1 |
| % (w/v) | | |
| PH | 7.4 | 7.3 |
| USP | Pass | Pass |
| EP-B | Pass | Pass |
| EP-A | Fail | Fail |
| | C. albicans 0.7 log | C. albicans 0.5 log |
| | reduction/7 days | reduction/7 days |
| | A. niger 0.8 log | A. niger 0.9 log |
| | reduction/7 days | reduction/7 days |

This example illustrates that the presence of both mannitol and a relatively small concentration of boric acid together have substantially no effect on the preservative efficacy of the composition. For example, when comparing Formulation 4 to Formulation 5, which contains 0.15% (w/v) of boric acid and 1% (w/v) of mannitol, it is shown that both formulations pass the USP and EP-B, but fail the EP-A.

### EXAMPLE 3

Two additional formulations are prepared and tested. A summary of the test results is as follows:

| Components | Formulation 6 | Formulation 7 |
|---|---|---|
| Oxy-chloro component | 50 | |
| (2) (ppm) | | |
| Pemulen TR-2 (1) | 0.05 | |
| % (w/v) | | |
| Castor oil | 1.25 | |
| % (w/v) | | |
| Mannitol | — | |
| % (w/v) | | |
| PH | 7.4 | |
| Purified Water | QS 100% | |
| Polysorbate 80% (w/v) | 1 | 0.8 |
| Boric acid | — | 0.2 |
| % (w/v) | | |
| Glycerin | 2.2 | 1 |
| % (w/v) | | |
| USP | Pass | Pass |
| EP-B | Fail | Pass |
| | C. albicans 0.4 log | |
| | reduction/14 days | |
| | A. niger 0.4 log reduction/ | |
| | 14 days | |
| EP-A | Fail | Fail |
| | S. aureus 1.2 log | A. niger 0.7 log |
| | reduction/6 hours | reduction/7 days |
| | C. albicans 0.4 log | |
| | reduction/7 days | |
| | A. niger 0.3 log | |
| | reduction/7 days | |

This example illustrates that without mannitol in the formulation, the presence of even small amounts of boric acid, together with the oxy-chloro component improves the preservative efficacy of Formulation 7. Thus, while Formulation 6, which includes no boric acid, fails both the EP-A and the EP-B test criteria, Formulation 7, which includes 0.2% (w/v) of boric acid, passes the EP-B test criteria.

### EXAMPLE 4

Two further formulations are prepared and tested. A summary of the test results is as follows:

| | | |
|---|---|---|
| Components | Formulation 8 | Formulation 9 |

| | |
|---|---|
| Oxy-chloro component | 150 |
| (2) (ppm) | |
| Pemulen TR-2 (1) | 0.1 |
| % (w/v) | |
| Polysorbate 80 | 1 |
| % (w/v) | |
| Castor Oil | 1.25 |
| % (w/v) | |
| Glycerin | 1 |
| % (w/v) | |
| PH | 7.3 |
| Purified Water | QS 100% |

| | | |
|---|---|---|
| Boric acid | 0.15 | 0.6 |
| % (w/v) | | |
| Mannitol | 1.5 | — |
| % (w/v) | | |
| USP | Pass | Pass |
| EP-B | Fail | Pass |
| | A. niger 0.2 log | |
| | reduction/14 days | |
| EP-A | Fail | Pass |
| | C. albicans 0.2 log | |
| | reduction/14 days | |
| | A. niger 0.1 log | |
| | reduction/14 days | |

This example illustrates that preservative efficacy is significantly enhanced in oxy-chloro component-containing formulations when boric acid is used, for example, at a concentration of 0.6% (w/v), and mannitol is not present. Formulation 9 passes all of the USP, EP-A and EP-B test criteria. In contrast, Formulation 8 passes only the USP test criteria.

### EXAMPLE 5

Another two formulations are prepared and tested. A summary of the test results is as follows:

| Components | Formulation 10 | Formulation 11 |
|---|---|---|
| Oxy-chloro component | 25 | 25 |
| (2) (ppm) | | |
| Pemulen TR-2 (1) | — | 0.1 |
| % (w/v) | | |
| Polysorbate 80 | — | 1 |
| % (w/v) | | |
| Castor oil | — | 1.25 |
| % (w/v) | | |
| Glycerin | — | 1 |
| % (w/v) | | |
| PH | 7.3 | 7.3 |
| Purified Water | QS 100% | QS 100% |
| Boric Acid | 0.6 | 0.6 |
| % (w/v) | | |
| Sodium Chloride % (w/v) | 0.3 | — |
| USP | (not run) | Pass |
| EP-B | Pass | Pass |
| EP-A | Fail | Pass |
| | S. aureus 1.3 log reduction/ | |
| | 6 hours | |
| | P. aeruginosa 0.7 log | |
| | reduction/6 hours | |

This example illustrates that the presence of glycerin in a formulation, including both oxy-chloro component and boric acid, further enhances the preservative efficacy of the composition. Formulation 11, which includes all of an oxy-chloro component, glycerin and boric acid passes all of the USP, EP-A and EP-B test criteria. Formulation 10 does not.

### EXAMPLE 6

A formulation is prepared and tested. A summary of the results of these tests is as follows:

| Components | Formulation 12 |
|---|---|
| Brimonidine tartrate | 0.15 |
| (3) % (w/v) | |
| Oxy-chloro component | 150 |
| (2) (ppm) | |
| Pemulen TR-2 (1) | 0.1 |
| % (w/v) | |
| Polysorbate 80 | 1 |
| % (w/v) | |
| Castor Oil | 1.25 |
| % (w/v) | |
| Glycerin | 1 |
| % (w/v) | |
| PH | 7.3 |
| Purified Water | QS 100% |
| Boric acid | 0.6 |
| % (w/v) | |
| Mannitol | — |
| % (w/v) | |

(3) 5-bromo-6-(2-imidozolin-2-ylamino) quinoxaline tartrate

This formulation passes all of the USP, EP-A and EP-B test criteria. In addition, the formulation is an effective composition, in the form of an oil-in-water emulsion, for delivering brimonidine to the eye of a human or animal.

While this invention has been described with respect to various specific examples and embodiments, it is to be understood that the invention is not limited thereto and that it can be variously practiced with the scope of the following claims.

What is claimed is:

1. An ophthalmic composition comprising a carrier component, a quinoxaline component present in an amount effective in providing a desired therapeutic effect to a human or animal to whom the composition is administered, an oxy-chloro component present at a concentration of greater than about 75 ppm and a borate component present in an amount effective to enhance a preservative efficacy of the composition relative to a substantially identical composition without the borate component.

2. The composition of claim 1 wherein the quinoxaline component is selected from the group consisting of quinoxalines, salts thereof and mixtures thereof.

3. The composition of claim 1 wherein the quinoxaline component is selected from the group consisting of 5-bromo-6-(2-imidozolin-2-ylamino) quinoxaline, salts thereof and mixtures thereof.

4. The composition of claim 1 wherein the oxy-chloro component comprises a chlorite component.

5. The composition of claim 1 wherein the oxy-chloro component is present in an amount in a range of greater than about 75 ppm to about 5000 ppm by weight of the composition.

6. The composition of claim 1 wherein the borate component is selected from boric acid, salts thereof and mixtures thereof.

7. The composition of claim 1 wherein the composition includes substantially no mannitol.

8. The composition of claim 1 which further comprises a glycerin component in an amount effective to further enhance the preservative efficacy of the composition.

9. The composition of claim 8 wherein the glycerin is present in an amount in a range of about 0.01% to about 10% (w/v) of the composition.

10. The composition of claim 1 wherein the composition comprises an aqueous component and an oily component.

11. The composition of claim 10 which is in the form of an oil-in water emulsion.

12. The composition of claim 11 wherein the aqueous component has a pH effective to produce a desired partitioning of the quinoxaline component between the aqueous component of the oily component.

13. The composition of claim 12 wherein the pH is between about 3.0 and about 9.0.

14. The composition of claim 12 wherein about 50% or more of the quinoxaline component is located in the aqueous component.

15. The composition of claim 12 wherein about 50% or more of the quinoxaline component is located in the oily component.

16. An ophthalmic composition comprising a carrier component, a therapeutic component present in an amount effective in providing a desired therapeutic effect to a human or animal to whom the composition is administered, an oxy-chloro component present at a concentration of greater than about 75 ppm and a borate component present in an amount effective to enhance a preservative efficacy of the composition relative to a substantially identical composition without the borate component.

17. The composition of claim 16 wherein the oxy-chloro component is present in an amount in a range of greater than about 75 ppm to about 5000 ppm by weight of the composition.

18. The composition of claim 16 wherein the borate component is selected from boric acid, salts thereof and mixtures thereof.

19. The composition of claim 16 wherein the composition includes substantially no mannitol.

20. The composition of claim 16 which further comprises a glycerin component in an amount effective to further enhance the preservative efficacy of the composition.

21. The composition of claim 16 wherein the glycerin is present in an amount in a range of about 0.01% to about 10% (w/v) of the composition.

22. The composition of claim 16 wherein the composition comprises an aqueous component and an oily component.

23. The composition of claim 22 which is in the form of an oil-in water emulsion.

24. The composition of claim 23 wherein the aqueous component has a pH effective to produce a desired partitioning of the therapeutic component between the aqueous component of the oily component.

25. An ophthalmic composition comprising a carrier component, a polyanionic component present in an amount effective to provide lubrication to an eye when the composition is administered to an eye, an oxy-chloro component present at a concentration of greater than 75 ppm and a borate component present in an amount effective to enhance a preservative efficacy of the composition relative to a substantially identical composition without the borate component.

26. The composition of claim 25 wherein the polyanionic component includes a material selected from the group consisting of anionic cellulosic derivatives and mixtures thereof.

27. The composition of claim 25 wherein the polyanionic component is selected from the group consisting of carboxy methyl celluloses and mixtures thereof.

28. The composition of claim 25 wherein the polyanionic component is present in an amount in a range of about 0.1% to about 5.0% (w/v) of the composition.

29. The composition of claim 25 wherein the polyanionic component includes a first polyanionic component portion having a first molecular weight, and a second polyanionic component portion having a different second molecular weight wherein the first molecular weight is greater than the second molecular weight, and the composition has an increased ability to adhere to an eye when the composition is administered to an eye relative to a substantially identical composition having an equal total amount of the polyanionic component and substantially no first polyanionic component portion.

30. The composition of claim 29 wherein the composition has a reduced ability to cause blurriness of vision in an eye when the composition is administered to an eye relative to a substantially identical composition having an equal total amount of polyanionic component and substantially no second polyanionic component portion.

31. The composition of claim 25 wherein the borate component is selected from boric acid, salts thereof and mixtures thereof.

32. The composition of claim 25 wherein the composition includes substantially no mannitol.

33. The composition of claim 25 which further comprises a glycerin component in an amount effective to further enhance the preservative efficacy of the composition.

34. The composition of claim 33 wherein the glycerin is present in an amount in a range of about 0.01% to about 10% (w/v) of the composition.

35. The composition of claim 25 wherein the composition comprises an aqueous component and an oily component.

36. The composition of claim 25 which is in the form of an oil-in water emulsion.

37. An ophthalmic composition comprising a carrier component, an oxy-chloro component present in an amount effective in preserving the composition and a glycerin component present in an amount effective to enhance a preservative efficacy of the composition relative to a substantially identical composition without the glycerin component.

38. The composition of claim 37 wherein the oxy-chloro component is present in an amount of at least about 0.002% (w/v) of the composition.

39. The composition of claim 37 which further comprises a therapeutic component present in an amount effective in providing a desired therapeutic effect to a human or animal to whom the composition is administered.

40. The composition of claim 39 wherein the therapeutic component is a quinoxaline component.

41. The composition of claim 40 wherein the quinoxaline component is selected from the group consisting of 5-bromo-6-(2-imidozolin-2-ylamino) quinoxaline, salts thereof and mixtures thereof.

42. The composition of claim 37 further comprising a polyanionic component in an amount effective to provide lubrication to an eye when the composition is administered to an eye.

43. The composition of claim 37 wherein the composition includes substantially no mannitol.

44. The composition of claim 37 wherein the composition comprises an emulsion having an aqueous component and an oily component.
